(19) United States
(12) Plant Patent Application Publication
(10) Pub. No.: US 2017/0118888 P1
(43) Pub. Date: Apr. 27, 2017

(54) Picea plant named 'Dougart 12'

(50) ;

(71) Applicant: Norbert Artmeyer, (US)

(72) Inventor: Andy Domen, Wernhout (NL)

(21) Appl. No.: 14/756,885

(22) Filed: Oct. 26, 2015

(51) Int. Cl. A01H 7/00

(52) U.S. Cl. PLT/213

(57) ABSTRACT

A new and distinct Picea abies plant that exhibits a compact and pyramidal growth habit.

Botanical classification: Picea abies (L.) H. Karst.

Varietal denomination: ‘Dougart12’.

### BACKGROUND OF THE INVENTION

The present invention comprises a new and distinct plant of Picea abies (L.) H. Karst. (Norway spruce) known by the varietal name ‘Dougart12’. The new variety was discovered and selected from a group of seedlings in a cultivated area in 2000 in Wernhout, The Netherlands. As such, its parental varieties are unknown. When compared to Picea abies variety ‘Will's Zwerg’ (unpatented), ‘Dougart12’ is more symmetrical in overall appearance, exhibits a faster growth rate, and has a smaller branch diameter. ‘Dougart12’ has been trial and field tested and has been found to retain its distinctive characteristics and remain true to type through successive propagations. The following characteristics distinguish the new variety from other Picea abies varieties:

1. Upright, pyramidal growth habit;

2. Compact (miniature) size;

3. Slow growth rate;

4. Short branches and internodes; and

5. Dark green-colored and short needles;

### DESCRIPTION OF THE DRAWINGS

The accompanying photographic drawings illustrate the new variety, with the color being as nearly true as is possible with color illustrations of this type:

FIG. 1 shows a 3 year old plant of the new variety in a container;

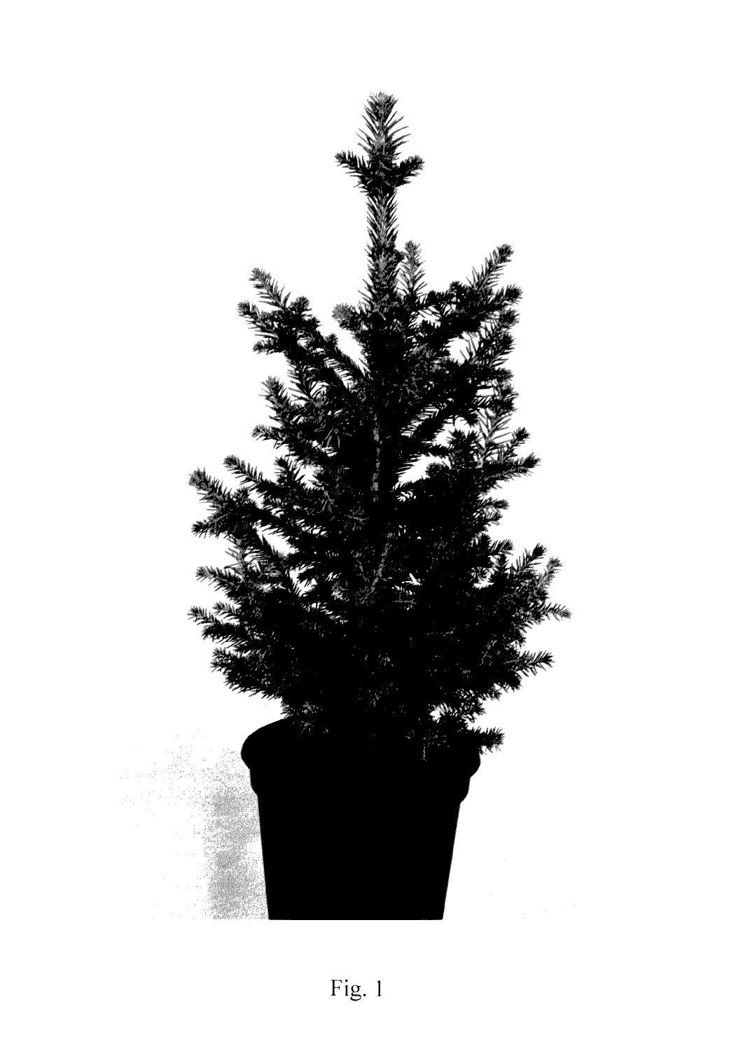

FIG. 2 shows a 4.5 year old plant of the new variety in a container;

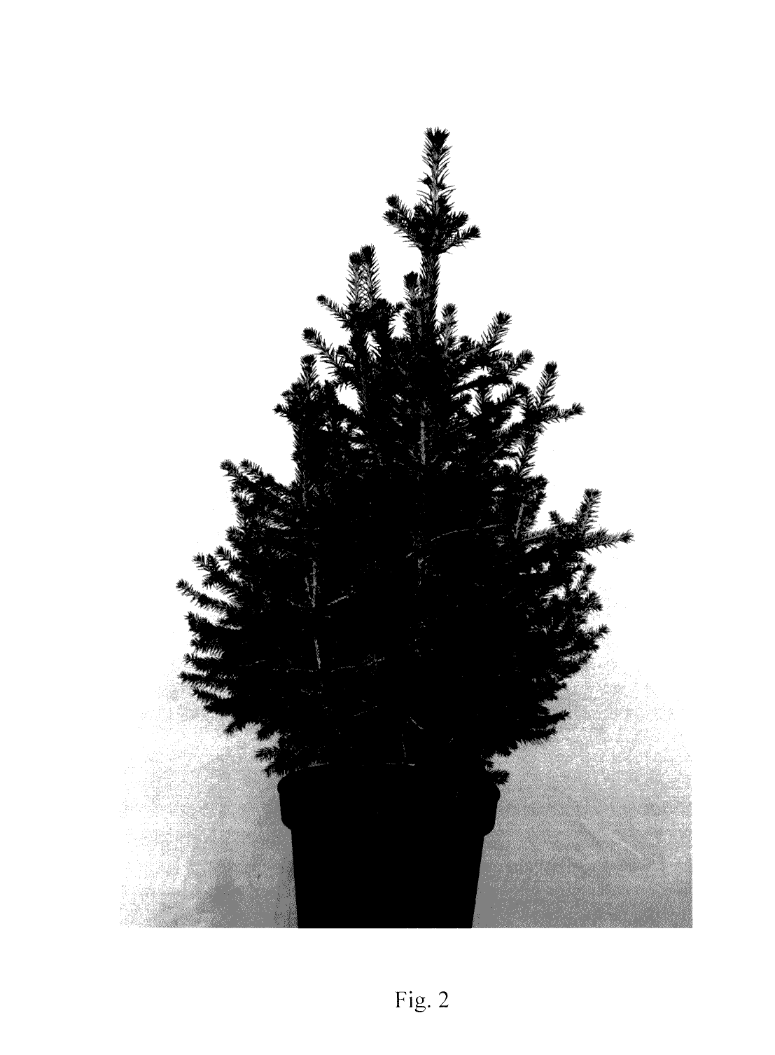

FIG. 3 shows an unpotted plant of the new variety;

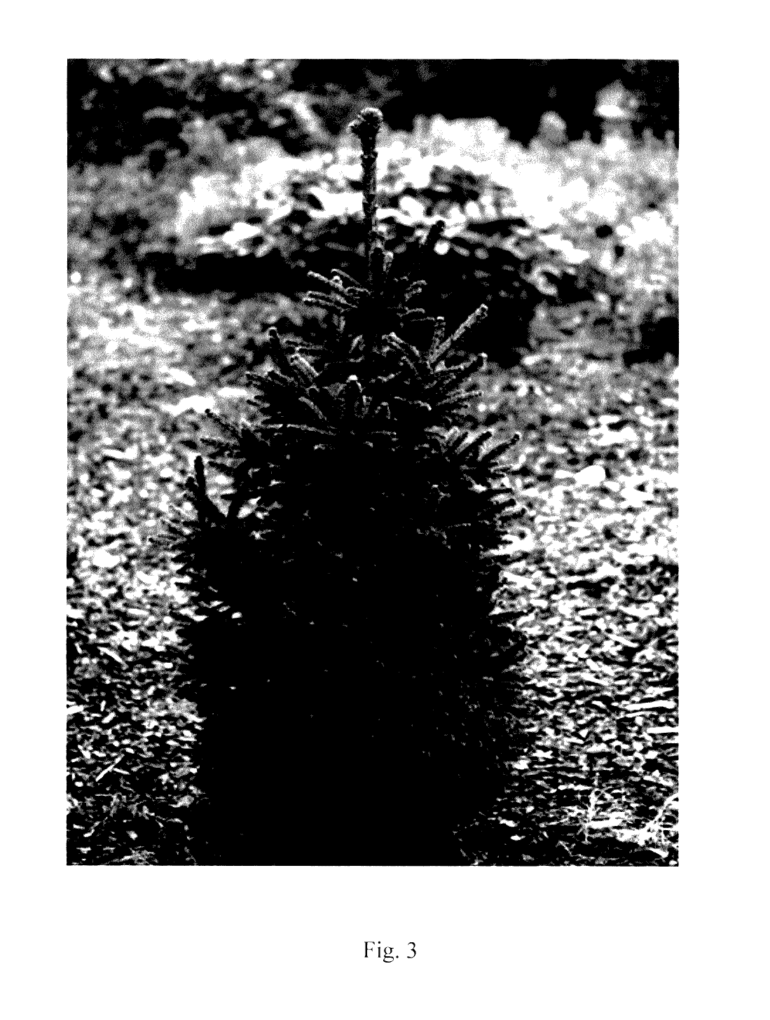

FIG. 4 shows a close-up view of the middle portion of the new variety;

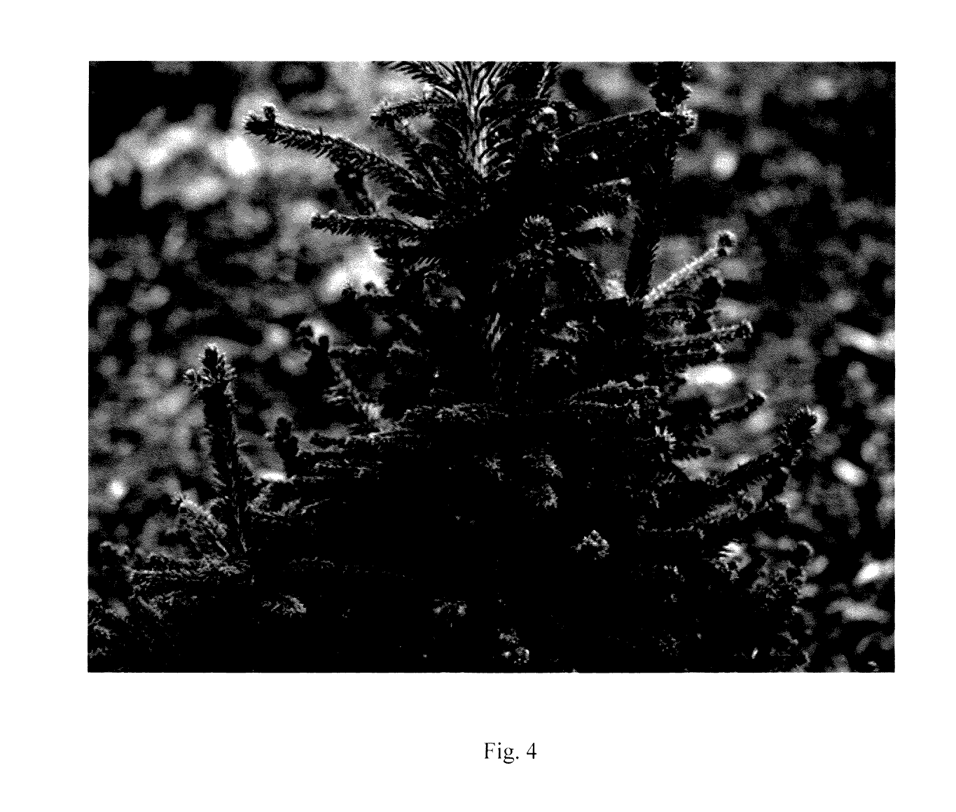

FIG. 5 shows a close-up view of the point of the new variety; and

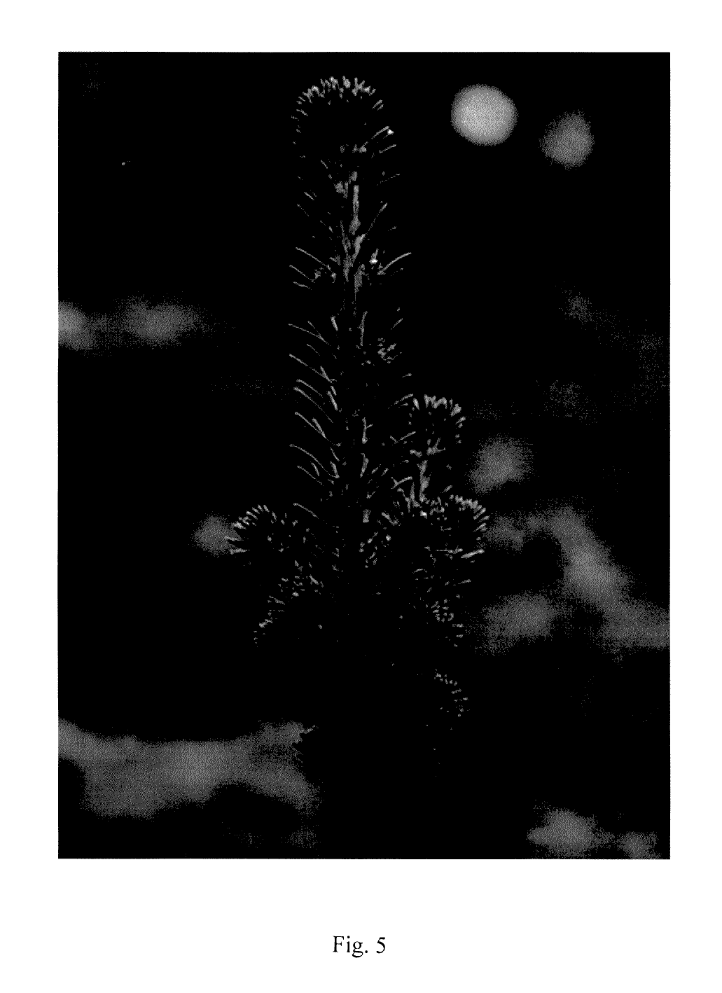

FIG. 6 shows a close-up view of a branch of the new variety.

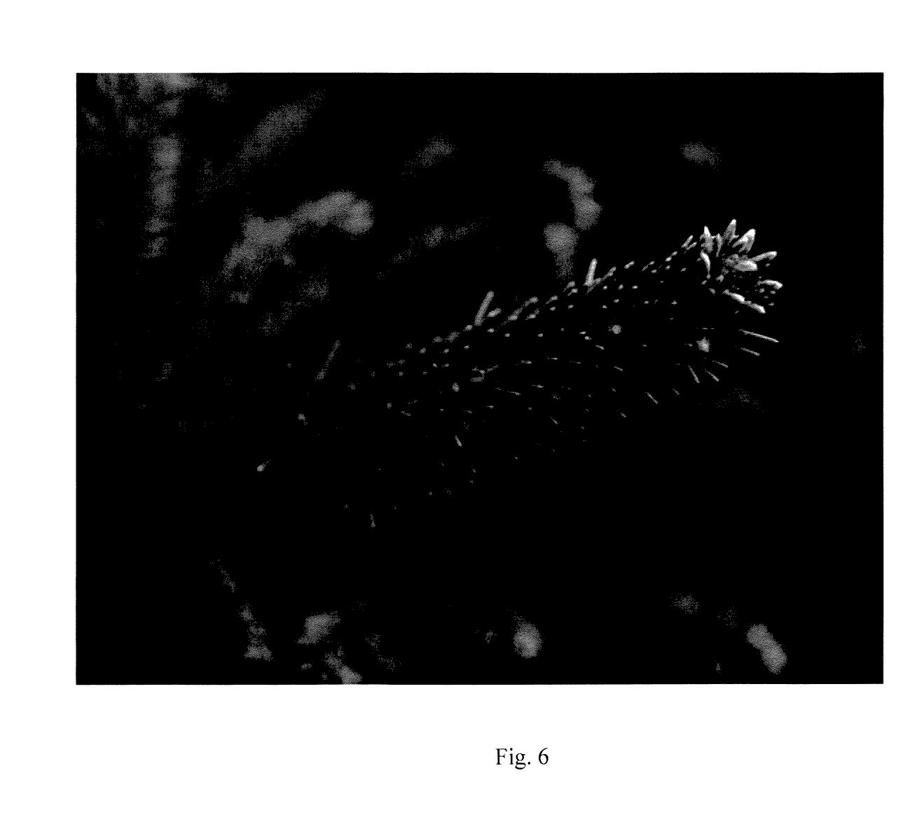

### DESCRIPTION OF THE PLANT

The following detailed description sets forth the characteristics of the new cultivar. The data that defines these characteristics were collected by asexual reproductions performed via cuttings in 2008 carried out in Hörstel-Riesenbeck, Germany. Plants of the new variety were grown under natural daylight and outdoors with full sun. The color readings and measurements were primarily taken on 4-year-old plants grown in 7.5 liter containers. Color references are primarily to the R.H.S. Colour Chart of The Royal Horticultural Society of London.
Plant:
   Plant type and use.—Coniferous, evergreen tree for use as a Christmas tree.
   Shape.—Upright and conical/pyramidal.
   Growth habit.—Upright and conical; the main trunk grows from the base and has lateral branches.
   Growth rate.—Average of 30 cm per season.
   Overall height.—200 cm at 10 years.
   Overall diameter.—118 cm at 10 years.
   Hardiness.—Typical of Picea abies plants.
   Cold temperature tolerance.—Typical of Picea abies plants, to −25° C.
   Time to produce a finished plant.—4 years, with a height of 60-70 cm.
   Disease/pest resistance/susceptibility.—None observed to date.
   Trunk.—Diameter: 2.2 cm, measured at 20 cm in height from the ground. Color: Grey-brown. Texture: Rough and scale-like.
Branch description:
   Average number.—Main branches: 1 trunk has 7 whorls and there are 4-5 main branches per whorl, averaging a total of 30 main branches when a plant is 50-60 cm high. Secondary branches: 4-6 per main branch. Twigs: 0-3 per secondary branch; low number of twigs overall.
   Shape.—Main branches: Rounded. Secondary branches: Rounded. Twigs: Rounded.
   Color.—Main branches: Greyed-Orange Group RHS 164B. Secondary branches: Greyed-Yellow Group RHS 162B. Twigs: Greyed-Yellow Group RHS 162B.
   Diameter.—Main branches: 0.5 cm. Secondary branches: 0.2 cm. Twigs: 0.15 cm.
   Length.—Main branches: 16-27 cm. Secondary branches: 4-8 cm. Twigs: 1-2 cm.
   Surface description.—Main branches: Rough and scale-like. Secondary branches: Rough and scale-like. Twigs: Rough and scale-like.
   Branching description.—Main branches extend upwardly from trunk at 30-45° angles.
   Internode length.—Between main and secondary branches: 3-4 cm. Between twigs: Very short, with a low number of twigs.
Needle description:
   Needles.—Arrangement: Alternate around the branches and twigs; imperfectly radial. Fragrance: Typical of Picea abies plants; spruce fragrance. Shape: Needle-shaped with weak curvature. Length: 1.2 cm. Width: 0.1 cm. Apex: Acute. Margin: Entire. Number per main branch: About 25 needles/3 cm. Number per secondary branch: About 40 needles/3 cm. Number per twig: About 20 needles/1 cm. Immature surface color: Green Group RHS 143A. Mature surface color: Green Group RHS 137A. Venation: None visible. Surface texture: Smooth. Resin glands: None visible.
Cone/seed description:
   Cone/seed description.—None observed.

I claim:

1. A new and distinct variety of Picea abies plant, as is herein illustrated and described.
